(19) United States
(12) Patent Application Publication
(10) Pub. No.: US 2004/0106199 A1
(43) Pub. Date: Jun. 3, 2004

(54) Charged cyclodextrin derivatives and their use in plant cell and tissue culture growth media

(76) Inventor: Alexey V. Eliseev, Brookline, MA (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/065,938

(22) Filed: Dec. 2, 2002

(51) Int. Cl. C12N 5/00; C12N 5/02

(52) U.S. Cl. 435/431

(57) ABSTRACT

The invention provides cyclodextrin derivatives that are substituted with groups bearing charges in aqueous solutions (charged cyclodextrins) in their salt forms and their use, also in combinations with other cyclodextrins, as useful components of plant cell and tissue growth media. The invention also comprises a new method of isolation of useful hydrophobic compounds, such as taxol, produced by plant cultures from the cyclodextrin-containing growth media and from the corresponding cell cultures.

### BACKGROUND OF INVENTION

Cyclodextrins are cyclic oligomers of glucose, in which the sugar moieties are linked with α-glycosidic bonds. Cyclodextrin molecules usually consist of six, seven, or eight sugar units (α-, β-, and γ-cyclodextrins, respectively). Cyclodextrin molecules are shaped as truncated cones and have internal cavities that are known to form inclusion complexes with hydrophobic compounds and moieties of comparable size (5 10 Å) in aqueous solutions. Due to their complexation properties, cyclodextrins have been widely used in pharmaceutical formulations, chromatography, deodorizing compositions, fabric treatment, etc. (for extensive review see J. Szejtli, Cyclodextrin Technology, Kluwer Acad. Publ., 1988).

It has been recently shown that cyclodextrins can be used as useful components of plant nutrient formulations increasing the growth of plant cells, as described in U.S. Pat. No. 6,087,176. It is believed that “cyclodextrins are useful in controlling solubility of insoluble components in the plant tissue culture medium. In addition, the cyclodextrins help adjust the osmolality of the medium to maintain proper turgor pressure in the cells.” (Column 4, line 64). Also cited are such effects as stabilizing biologically active and volatile substances in the media, protecting against the oxidation, and the increase in production of secondary metabolites.

One of the most pharmaceutically important plant growth processes is production of taxol and other bioactive taxanes in taxus cells. Taxol is extremely effective against refractory ovarian cancers, as well as breast and other cancers, and has been pronounced as a breakthrough in chemotherapy. Production of taxol from natural sources is extremely expensive, for example it takes three to six 100 year old Pacific yews to isolate the amount of drug needed for the treatment of one patient (see U.S. Pat. No. 5,407,816). Complete chemical synthesis of taxol is highly complex and has so far been only accomplished in a few academic laboratories as a result of many years of research (see e.g. R. A. Holton, et al., J. Am. Chem. Soc. 1994, 116(4), 1597-1598; K. C. Nicolaou, et al. Nature, 1994, 367(6464), 630-634). Production of taxol in plant cell culture processes is an important alternative approach, as described in a number of patents, e.g. U.S. Pat. No. 5,019,504; U.S. Pat. No. 5,407,816; U.S. Pat. No. 6,365,407. Therefore, there are apparent needs in further optimization of both the taxus cell culture media and methods of isolation of taxol from the cells to improve its production process.

### SUMMARY OF INVENTION

Plant cell and tissue growth media, including the media used for cultivation of taxus cells, contain multiple inorganic salt components that supply plants with such essential nutrients as potassium, ammonium, nitrate, and phosphate ions. Therefore, one of the approaches to media optimization is to develop an efficient combination of various components. The present invention addresses the above-identified need by providing cyclodextrin derivatives that are substituted with groups bearing charge in aqueous solutions (charged cyclodextrins) in their salt forms and their use, optionally in combinations with other cyclodextrins, as useful components of plant cell and tissue growth media and hydroponic solutions. The advantages of using charged cyclodextrins include their improved complexation properties toward other nutrients and cell metabolites, their usability in the salt forms with essential nutrient ions, and reduced osmolalities of the media. In addition, cyclodextrin phosphates are also capable of slowly releasing inorganic phosphate upon degradation, thus providing sustained release of this essential nutrient.

The present invention also comprises a new method of isolation of useful hydrophobic compounds, such as taxol, produced by plant cultures from the cyclodextrin-containing growth media and from the overall content of the corresponding cell cultures. This method is based on the separation of complexes of taxol and similar hydrophobic compounds from the low molecular weight components, such as salts, by size exclusion chromatography. The method is applicable to all types of cyclodextrins, although charged cyclodextrins are preferred because of their higher solubility in aqueous solutions.

### DETAILED DESCRIPTION

Definitions:

Guest molecules—small molecules, typically of hydrophobic nature, capable of forming non-covalent complexes with cyclodextrins in aqueous solutions. The complexes are typically formed through inclusion of all or part of the guest molecule into the cyclodextrin cavity. In the context of this invention, the guest molecules are typically represented by organic components of plant growth media and by essential plant metabolites, such as taxanes.

Charged cyclodextrins—cyclodextrins, modified with covalently attached substituents capable of bearing positive (cationic cyclodextrins) or negative (anionic cyclodextrins) charge in aqueous solutions.

Plants whole plants, plant organs, such as stems, leaves, stems, roots, flowers, meristematic tissue, seeds, yeasts, fungi, algae, plant tissue culture cells derived from any plant organ or tissue and progeny of same.

Charged cyclodextrins offer a number of advantages as components of plant nutrition formulations and plant cell culture media in comparison with unsubstituted cyclodextrins and other uncharged cyclodextrin derivatives, such as hydroxypropyl cyclodextrins, available commercially and described in the literature. The molecules of charged cyclodextrins contain hydrophobic cavites which form inclusion complexes with lipophilic small molecules in aqueous solutions. In addition, they contain one or more of hydrophilic side chains bearing charge, and therefore form non-covalent complexes with oppositely charged guest molecules. The combination of hydrophobic cavity and charged groups yields synergistic effect in formation of non-covalent complexes of charged cyclodextrins with amphiphilic organic ions, for example. Guest molecules involved in the formation of such complexes include multiple essential organic nutrients, such as vitamins and growth factors, as well as metabolites of plant cultures. The complex formation leads to increased solubility of the nutrients and metabolites in plant growth and cell culture media, their improved transport across biological membranes and can result in increased cell culture growth rates. Some examples of charged cyclodextrin nutrient combinations are listed below:

Cationic cyclodextrins, such as those substituted with ammonium and alkylammonium groups, form complexes with thiamine pyrophosphate mono-, and triphosphates, nicotinic acid adenine dinucleotide, nicotinic acid mononucleotide, riboflavin phosphate, riboflavin acetyl phosphate, flavin adenine mono- and dinucleotides, pyridoxal phosphate, biotin 4-amidobenzoic acid, 5-(N-biotinyl)-3 aminoallyl)-uridine 5″-triphosphate, inositol monophosphate, D-myo-inositol 1,4-bisphosphate, DL-myo-inositol 1,2-cyclic monophosphate, inositol hexaphosphate, myo-inositol hexasulfate, myo-inositol 2-monophosphate, D-myo-inositol 1-monophosphate, DL-myo-inositol 1-monophosphate, D-myo-inositol triphosphate, phenylacetic acid, benzoic acid, and gibberellins (e.g. GA1, GA2, GA3, GA4, GA7, GA38 etc.), for example.

Anionic cyclodextrins, such as those cyclodextrin phosphates, sulfates, succinylates, carboxymethyl cyclodextrins, form complexes with benzyl adenine, zeatin riboside, zeatin, isopentenyl adenine, indoleacetic acid, indole ethanol, indoleacetaldehyde, indoleacetonitrile, and the like.

In the context of this invention, it is of note that charged cyclodextrins form inclusion complexes with essential organic products secreted by plant cells into the extracellular media. In particular, such complexes are formed with taxol and other bioactive taxanes, which can be used for improved production and isolation of the latter, as shown below.

Charged cyclodextrins and their salts also act as important ionic components of plant growth media. Charged cyclodextrins can be synthesized and used in the salt forms with counterions that constitute essential inorganic plant nutrients. Such is the case, for example with potassium and ammonium salts of cyclodextrin phosphates and carboxylates, as well as with nitrate, phosphate, and sulfate salts of cyclodextrins substituted with ammonium groups, which provide sources for potassium, nitrogen, phosphorus and sulfur nutrition components.

In the case of charged cyclodextrins that bear more than one charged group, for example cyclodextrin bisphosphates, the nutrient counterions, for example potassium or ammonium, can be introduced in the growth media so as to decrease the osmotic pressure of the media, as compared to equivalent amounts of the corresponding inorganic salts. The decrease in osmotic pressure results from the fact that the multiple charged groups attached to a single cyclodextrin molecules yield as much contribution in the total osmolality, as a single species.

In addition to the above mentioned effects, cyclodextrin phosphates also undergo slow hydrolysis in aqueous solutions, leading to a release of inorganic phosphate that serves as an essential nutrient for plants. Such a hydrolysis process is catalyzed by plant phosphatases and other enzymes. Thus, cyclodextrin phosphates provide gradual regeneration of phosphate in plant growth media to compensate for the phosphate consumed by the plants.

Charged cyclodextrin derivatives can be synthesized by a variety of methods known from the literature by derivatization of unsubstituted cyclodextrins. The derivatization process usually involves substitution of one or more hydroxyl groups with activating agents, e.g. tosyl chloride, mesyl chloride, phosphoryl chloride, etc. followed by conversion of the activated (e.g. tosylated) positions into ionogenic groups. Most of the known derivatization techniques lead to the formation of mixtures of cyclodextrin derivatives, with varying degrees of modification and positions of the substituents on the cyclodextrin molecule. The composition and properties of such mixtures may vary depending on the deviations in the precise experimental protocol. For better control and more reproducible results while using charged cyclodextrins in plant growth media, it is preferred to obtain derivative(s) with controlled degree and mode of substitution with ionogenic groups. It is preferable to use the derivatives that are isolated and identified as individual compounds, e.g. those that contain a single substituent representing a charged group at a specific position of the cyclodextrin molecule, preferably the 6^{A} site at the upper rim of the cavity, as shown in Scheme 1. It is also preferred to isolate the charged cyclodextrin in a specific salt form so that it can be use as a plant nutrient. We describe here a general procedure of synthesis and isolation of such salt forms which represents an improvement or methods used in the literature in that it yields individual cyclodextrin derivatives in their salt forms that can be used as a plant nutrient.

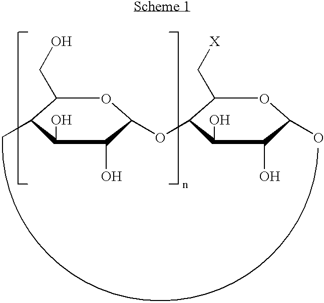

n=5-8, X charged group

### EXAMPLES

n=7, X=OPO₃(H)₂K-γ-cyclodextrin-6^{A}-monoposphate monopotassium salt.

n=7, X=OCO(CH₂)₂COOK-γ-cyclodextrin-6^{A}-monosuccinylate monopotassium salt.

n=6, X=NH₃NO₃-6^{A}-Deoxy-6^{A}-ammonium-β-cyclodextrin nitrate.

Example 1 describes an improved and modified procedure derived from the one used previously for synthesis of β-cyclodextrin-6^{A}-monoposphate (A. Cho, et al. Org. Lett. 2000, 2(12), 1741-1743).

### Example 1

### γ-cyclodextrin-6^{A}-monoposphate Monopotassium Salt

2.28 grams (1.76 mmol) of γ-cyclodextrin is dried at 80° C. for 3 days under vacuum (0.1 mm Hg). 70 ml of trimethyl phosphate is dried using molecular sieves for 3 days at 80° C. γ-cyclodextrin is flushed with argon, and the hot trimethyl phosphate is added by calumet. The resulting cloudy solution clears up after stirring for 30 minutes. The mixture is then cooled down to −15° C., and 500 μl (5.28 mmol) of phosphoryl chloride is added slowly. The reaction is left to run for 1 hour, and then quenched with 0.5 ml of distilled water. 150 ml of cold ether and then 100 ml of reagent grade acetone is added to precipitate the product. The precipitate is then filtered through a glass filter to give 4 g of white crystalline crude product. The crude product is then redissolved in 10 ml of distilled water and loaded on to a 24 cm by 3 cm anion exchange column filled with Q-Sepharose (Sigma). The column is first washed with 1 l of distilled water to remove most of the unreacted γ-cyclodextrin. The product is then eluted out with a gradient of 0-0.33 M aqueous ammonium hydrogen carbonate. The eluent speed is set to approximately 8 ml/min and the product elutes out approximately between 0.09M to 0.18M ammonium hydrogen carbonate concentration. The product presence in the chromatographic fractions is checked by thin layer chromatography (TLC) using a mixture of 70% ethanol in water and 7% ammonium hydroxide in water in the ratio 8:2 as the eluent. TLC plates are developed by burning with 10% sulfuric acid in methanol. The product has an R_{f} of 0.40 compared to R_{f} of 0.65 for γ-cyclodextrin. Lyophilization of the fractions yields 260 mg (12%) of γ-cyclodextrin-6^{A}-monoposphate monoammonium salt. The product is then redissolved in 200 ml of water, mixed with 2 g of pre-swollen Dowex HCR-W2 cation exchange resin (K⁺ form), stirred for 1 h, filtered, and lyophilized.

In the above procedure, additional fractions may be collected which elute from the Q-Sepharose column between 0.18M and 0.28M ammonium hydrogen carbonate. After their treatment according to the rest of the above procedure, these fractions are converted, via potassium ion exchange as described above into a mixture of cyclodextrin-6-bisphosphate dipotassium salts in an overall yield of ca. 20%.

The procedure described in Example 1 can be also used to make corresponding derivatives of α- and β-cyclodextrins.

### Example 2

### γ-cyclodextrin-6^{A}-monosuccinylate Monopotassium Salt

γ-Cyclodextrin (12 g, 9.25 mmol) dried, as described in Example 1, is added to 80 ml of dry pyridine under extensive stirring within 20 minutes. The solution is then quickly cooled down to 0° C. and succinic anhydride (812 mg, 8.12 mmol) is slowly added. The reaction mixture is stirred in an argon atmosphere for three days. After removing the solvent on rotary evaporator, the residue is dried at 50-60° C. using an oil pump for 2 days. The residue is then redissolved in 300 ml of water, mixed with 50 ml of pre-swollen beads of Dowex 50 WX2 (NH₄⁺ form) and stirred for 30 min. After filtration of the beads, the filtrate is lyophilized, and purified by ion exchange chromatography on 500 ml of Q-Sepharose (Sigma), eluting with the gradient of 0-0.5 M aqueous ammonium hydrogen carbonate. Cyclodextrin-containing fractions eluted in 0.5-1.5 M salt are collected and lyophilized yielding 5.55 g (42%) of analytically pure ammonium salt of γ-cyclodextrin-6^{A}-monosuccinylate. The product is then redissolved in 200 ml of water, mixed with 10 g of pre-swollen Dowex HCR-W2 cation exchange resin (K⁺ form), stirred for 1 h, filtered, and lyophilized.

The procedure described in Example 2 can be also used to make corresponding derivatives of α- and β-cyclodextrins.

Synthesis of cylodextrins substituted with ammonium groups (amino cyclodextrins) is performed as described in the literature. For the use of amino cyclodextrins in plant growth media, it is preferable to isolate their monosubstituted derivatives in salt form with useful counterions, such as nitrate, phosphate, or sulphate using corresponding anion exchange resins.

Charged cyclodextrins can be used in the plant growth media as additives used for overall growth acceleration, introduction of essential nutrients, slow release of certain nutrients, such as inorganic phosphate ions, as well as for the subsequent isolation of essential cell metabolites. While the above uses may be applied to a variety of plant cell and tissue growth media, of particular importance is their use for production of taxol and bioactive taxanes in taxus cell cultures.

### Example 3

The following medium composition is usable for the callus cultures of Taxus wallichiana, suc as those described in U.S. Pat. No. 6,365,407 B1 (amounts are given in mg/100 ml solution): β-cyclodextrin-6^{A}-monoposphate monopotassium salt (1250); 6^{A}-Deoxy-6^{A}-ammonium-β-cyclodextrin nitrate (1200); β-cyclodextrin-6^{A}-monoposphate monoammonium salt (200); potassium nitrate (150); magnesium sulfate heptahydrate (25), sodium dihydrogen phosphate hydrate (15); calcium chloride dihydrate (15); EDTA disodium salt (3.7); ferrous sulfate heptahydrate (2.8), boric acid (0.3); cobalt dichloride hexahydrate (0.0025); cupric sulfate pentahydrate (0.0025), manganese sulfate hydrate (1.0), zinc sulfate heptahydrate (0.2); potassium iodide (0.075); sodium molybdate dihydrate (0.025), myo-inositol (10), nicotinic acid (0.1), pyridoxine hydrochloride (0.1); thiamine hydrochloride (1.0), sucrose (2000).

### Example 4

The following medium composition is usable for cultivation of Taxux chinensis culture, such as one described in U.S. Pat. No. 5,407,816 (amounts are given in mg/100 ml solution):

β-cyclodextrin-6^{A}-monoposphate monopotassium salt (800); 6^{A}-Deoxy-6^{A}-ammonium-β-cyclodextrin nitrate (800); ammonium sulfate (3.35); boric acid (0.075); calcium chloride dihydrate (8.75); cobalt chloride hexahydrate (0.0006); cupric sulfate pentahydrate (0.0006); EDTA disodium salt dihydrate (0.93); ferrous sulfate heptahydrate (0.70); magnesium sulfate (3.1); manganese sulfate hydrate (2.25); sodium molybdate dihydrate (0.0062); potassium iodide (0.018); potassium phosphate (1.0) sodium dihydrogen phosphate (3.26); zinc sulfate heptahydrate (0.05); myo-inositol (12.5); nicotinic acid (0.075); pyridoxine hydrochloride (0.025); thiamine hydrochloride (0.35); sodium acetate (1.0); sucrose (4000); N6-benzyladenine (0.2); ascorbic acid (5.0); casein hydrolysate (50).

Charged cyclodextrins can also be used in plant cell and tissue growth media in combination with other cyclodextrins and their derivatives.

Addition of charged cyclodextrins to plant growth media can be also used for improved isolation of essential products of plant cells, such as taxol. As shown in U.S. Pat. No. 5,407,816, significant amounts of taxol and other bioactive taxanes are secreted into extracellular media during the growth of taxus cell cultures. These secreted compounds contain hydrophobic moieties, such as side chain phenyl rings of the taxol molecule. Compounds of such structure are known to form particularly strong complexes with cyclodextrins, as has been demonstrated, for example for taxol complexes with unsubstituted β-cyclodextrin. When charged cyclodextrins are present in the growth media, they form complexes with the hydrophobic secreted compounds, particularly when the cyclodextrins are present in large excess over the secreted compounds, as in examples 3 and 4. Upon separation of cells from the growth medium, complexes of secreted compounds, such as taxanes, can be isolated from other medium components, i.e. salts, organic nutrients, growth factors, etc., via size exclusion chromatography, as described in the following example.

### Example 5

The cell culture Taxus chinensis is grown in the medium described in Example 4. After 9 days, the cells are separated from the medium by suction filtration, and the filtrate is lyophilized. The dry residue is then redissolved in 3-5 mL water per 100 mL of original filtrate and loaded onto a size exclusion column filled with 100-300 ml of pre-swollen Sephadex G10 or Biogel P2. Elution is performed with water at a high flow rate (10-20 ml/min). Cyclodextrin-containing fractions which elute prior to other growth medium components, are detected by a polarimetry detector and collected. Taxol and other taxanes are then separated from cyclodextrins by extraction into an organic solvent.

The above isolation procedure based on size exclusion separation is particularly suitable for isolation of taxanes of higher purity than usually achieved in direct extraction methods, such as described in U.S. Pat. No. 5,019,504. One of the reasons is that taxol and other taxanes form complexes with more than one cyclodextrin molecule due to inclusion of two or more side chain phenyl rings into cavities of different cyclodextrin molecules. This results in the formation of high molecular weight complexes that are separated by size exclusion from other organic media components.

The isolation procedure based on size exclusion separation can be also applied to any other cyclodextrin containing media, such as those previously described in U.S. Pat. No. 6,087,176. The use of charged cyclodextrins is preferred, because their high aqueous solubility allows one to use high cyclodextrin concentrations in the sample loaded onto the size exclusion column. This results in low sample volumes, and prevents dissociation of cyclodextrin-taxol complexes on the column, which improves separation from other medum components.

The isolation procedure based on size-exclusion separation can be also applied to isolate the hydrophobic constituents of the cell and tissue cultures grown in the cyclodextrin-containing media. In that case the cultures are homogenized by sonication, grinding, or any other technique destroying the cell membranes prior to the isolation step.

1. A composition of matter, comprising a monopotassium salt of cyclodextrin-6^{A}-monophosphate.

2. The composition of claim 1, wherein the monopotassium salt of cyclodextrin-6^{A}-phosphate is selected from the group consisting of monopotasium α-cyclodextrin-6^{A}-monophosphate, monopotassium β-cyclodextrin-6^{A}-monophosphate, monopotassium γ-cyclodextrin-6^{A}-monoposphate and mixtures thereof.

3. A composition of matter, comprising a monopotassium salt of cyclodextrin-6^{A}-monosuccinylate.

4. The composition of claim 3, wherein the monopotassium salt of cyclodextrin-6^{A}-monosuccinylate is selected from the group consisting of monopotasium α-cyclodextrin-6^{A}-monosuccinylate, monopotassium β-cyclodextrin-6^{A}-monosuccinylate, monopotassium γ-cyclodextrin-6^{A}-monosuccinylate and mixtures thereof.

5. A growth medium for plant cell or tissue cultures, comprising at least one charged cyclodextrin.

6. The growth medium of claim 5, wherein at least one charged cyclodextrin is selected from the group consisting of the monopotassium salt of cyclodextrin-6^{A}-monophosphate, monopotasium α-cyclodextrin-6^{A}-monophosphate, monopotassium β-cyclodextrin-6^{A}-monophosphate, monopotassium γ-cyclodextrin-6^{A}-monoposphate, monoammonium salt of cyclodextrin-6^{A}-monophosphate, monoammonium α-cyclodextrin-6^{A}-monophosphate, monoammonium β-cyclodextrin-6^{A}-monophosphate, monoammonium γ-cyclodextrin-6^{A}-monoposphate, monopotassium salt of cyclodextrin-6^{A}-monosuccinylate, monopotasium α-cyclodextrin-6A-monosuccinylate, monopotassium β-cyclodextrin-6^{A}-monosuccinylate, monopotassium γ-cyclodextrin-6^{A}-monosuccinylate, monoammonium salt of cyclodextrin-6^{A}-monosuccinylate, monoammonium α-cyclodextrin-6^{A}-monosuccinylate, monoammonium β-cyclodextrin-6^{A}-monosuccinylate, monoammonium-γ-cyclodextrin-6^{A}-monosuccinylate,6^{A}-deoxy-6^{A}-ammonium-α-cyclodextrin nitrate, 6^{A}-deoxy-6^{A}-ammonium-β-cyclodextrin nitrate, 6^{A}-deoxy-6^{A}-ammonium-γ-cyclodextrin nitrate, 6^{A}-deoxy-6^{A}-ammonium-α-cyclodextrin sulfate, 6^{A}-deoxy-6^{A}-ammonium-β-cyclodextrin sulfate, 6^{A}-deoxy-6^{A}-ammonium-γ-cyclodextrin sulfate, 6^{A}-deoxy-6^{A}-ammonium-α-cyclodextrin phosphate, 6^{A}-deoxy-6^{A}-ammonium-β-cyclodextrin phosphate, 6^{A}-deoxy-6^{A}-ammonium-γ-cyclodextrin phosphate and mixtures thereof.

7. The growth medium of claim 5, further comprising compounds selected from the group consisting of α-cyclodextrin, β-cyclodextrin, γ-cyclodextrin, their non-ionic derivatives and mixtures thereof, the derivatives containing substituents at positions 2, 3, and 6 of the glucose residues.

8. The growth medium of claim 7, wherein the substituents are selected from the group consisting of hydroxypropyl groups, alkyl, acyl, alkylsulphonyl, and mixtures thereof.

9. A method of growing plant cell or tissue cultures, comprising contacting plant cell or tissue cultures from the genus taxus with a growth medium comprising at least one charged cyclodextrin.

10. The method of claim 9, wherein the charged cyclodextrin is selected from the group consisting of the monopotassium salt of cyclodextrin-6^{A}-monophosphate, monopotasium α-cyclodextrin-6^{A}-monophosphate, monopotassium β-cyclodextrin-6^{A}-monophosphate, monopotassium γ-cyclodextrin-6^{A}-monoposphate, monoammonium salt of cyclodextrin-6^{A}-monophosphate, monoammonium α-cyclodextrin-6^{A}-monophosphate, monoammonium β-cyclodextrin-6^{A}-monophosphate, monoammonium γ-cyclodextrin-6^{A}-monoposphate, monopotassium salt of cyclodextrin-6^{A}-monosuccinylate, monopotasium α-cyclodextrin-6A-monosuccinylate, monopotassium β-cyclodextrin-6^{A}-monosuccinylate, monopotassium γ-cyclodextrin-6^{A}-monosuccinylate, monoammonium salt of cyclodextrin-6^{A}-monosuccinylate, monoammonium α-cyclodextrin-6A-monosuccinylate, monoammonium β-cyclodextrin-6^{A}-monosuccinylate, monoammonium-γ-cyclodextrin-6^{A}-monosuccinylate,6^{A}-deoxy-6^{A}-ammonium-α-cyclodextrin nitrate, 6^{A}-deoxy-6^{A}-ammonium-β-cyclodextrin nitrate, 6^{A}-deoxy-6^{A}-ammonium-γ-cyclodextrin nitrate, 6^{A}-deoxy-6^{A}-ammonium-α-cyclodextrin sulfate, 6^{A}-deoxy-6^{A}-ammonium-β-cyclodextrin sulfate, 6^{A}-deoxy-6^{A}-ammonium-γ-cyclodextrin sulfate, 6^{A}-deoxy-6^{A}-ammonium-α-cyclodextrin phosphate, 6^{A}-deoxy-6^{A}-ammonium-β-cyclodextrin phosphate, 6^{A}-deoxy-6^{A}-ammonium-γ-cyclodextrin phosphate and mixtures thereof.

11. The method of claim 9, further comprising contacting plant cell or tissue cultures from the genus taxus with a growth medium further comprising compounds selected from the group consisting of α-cyclodextrin, β-cyclodextrin, γ-cyclodextrin, their non-ionic derivatives and mixtures thereof, the derivatives containing substituents at positions 2, 3, and 6 of the glucose residues.

12. The method of claim 11, wherein the substituents are selected from the group consisting of hydroxypropyl groups, alkyl, acyl, alkylsulphonyl, and mixtures thereof.

13. A method of isolating hydrophobic compounds produced by plant cell or tissue cultures, the plant cell or tissue cultures growing in cyclodextrin containing media, the method comprising separating at least one cylcodextrin complex with at least one hydrophobic compound by size exclusion chromatography, followed by dissociating of the at least one cyclodextrin complex.

14. The method of claim 13, wherein the at least one hydrophobic compound is secreted by at least one plant cell into extracellular media.

15. The method of claim 13, wherein the at least one hydrophobic compound is at least one bioactive taxane.

16. The method of claim 15, wherein the bioactive taxane is taxol.

17. A composition of matter, comprising a salt of a cationic cyclodextrin of a plant nutrient.

18. The composition of claim 17, wherein the plant nutrient is selected from the group consisting of nitrate, sulfate, phosphate, and mixtures thereof.

19. The composition of claim 17, wherein the cationic cyclodextrin is selected from the group consisting of ammonium cyclodextrin, alkylammonium cyclodextrin, and mixtures thereof.

20. A composition of matter, comprising a dipotassium salt of cyclodextrin-6-bisphosphate.

21. The composition of claim 20, wherein the dipotassium salt of cyclodextrin-6-bisphosphate is selected from the group consisting of dipotassium salt of α-cyclodextrin-6-bisphosphate, dipotassium salt of β-cyclodextrin-6-bisphosphate, dipotassium salt of γ-cyclodextrin-6-bisphosphate and mixtures thereof.
